(19) United States
(12) Patent Application Publication
(10) Pub. No.: US 2014/0349244 A1
(43) Pub. Date: Nov. 27, 2014

(54) SYRINGE SLEEVE

(71) Applicant: Rakesh Patel, Alta Loma, CA (US)

(72) Inventor: Rakesh Patel, Alta Loma, CA (US)

(21) Appl. No.: 13/900,958

(22) Filed: May 23, 2013

(51) Int. Cl. A61C 1/08; A61M 5/178

(52) U.S. Cl. CPC A61C 1/088; A61M 5/178
USPC 433/29

(57) ABSTRACT

A sleeve may be attached to a syringe. The sleeve may include a hollow tube configured to slide on, or attach to, a syringe. The sleeve may also include a plurality of lighting devices located near a distal end of the hollow tube. The plurality of lighting devices may be configured to illuminate when the sleeve is activated.

### FIELD

The present invention relates to a sleeve and, more particularly, to a sleeve attached to a syringe that includes lighting devices.

### BACKGROUND

When a medical professional, such as a dentist, (hereinafter “user”) is injecting anesthetic within the mouth of a patient, the user must follow certain parameters with respect to the location point of the injection of the needle. In order to increase accuracy to be closer to the location point, the user may use an external light positioned above the patient and the user. However, this external light may cause a shadow to fall over the patient's mouth when the user, or his or her hands, are situated near or above the patient's mouth. This may make it more difficult for the user to accurately inject the anesthetic near the location point, requiring the user to re-inject the anesthetic a subsequent time. Thus, an improved approach to illumination may be beneficial.

### SUMMARY

Certain embodiments of the present invention may provide solutions to the problems and needs in the art that have not yet been fully identified, appreciated, or solved by current syringes. For example, some embodiments of the present invention pertain to an illuminating sleeve configured to slide on, or attach to, a syringe. The sleeve increases the accuracy of the user by improving visibility of the penetration point of the needle and reducing the need for re-injection when the user is injecting anesthetics in the gums of a patient's mouth.

In one embodiment, an apparatus is provided. The apparatus includes a hollow tube configured to slide on, or attach to, a syringe. The apparatus also includes a plurality of lighting devices located proximate to a distal end of the hollow tube. The plurality of lighting devices are configured to illuminate when the apparatus is activated.

In another embodiment, an apparatus is provided. The apparatus includes a sleeve configured to attach to, or slide on to, a syringe. The apparatus also includes a plurality of lighting devices configured to illuminate when the sleeve is activated.

### BRIEF DESCRIPTION OF THE DRAWINGS

In order that the advantages of certain embodiments of the invention will be readily understood, a more particular description of the invention briefly described above will be rendered by reference to specific embodiments that are illustrated in the appended drawings. While it should be understood that these drawings depict only typical embodiments of the invention and are not therefore to be considered to be limiting of its scope, the invention will be described and explained with additional specificity and detail through the use of the accompanying drawings, in which:

FIG. 1 illustrates a conventional dental syringe.

FIGS. 2A and 2B illustrate a syringe sleeve, according to an embodiment of the present invention.

FIG. 3 illustrates a syringe with a syringe sleeve, according to an embodiment of the present invention.

FIG. 4 illustrates a syringe with a partial syringe sleeve, according to an embodiment of the present invention.

FIG. 5 illustrates a syringe with a half syringe sleeve, according to an embodiment of the present invention.

FIG. 6 is a block diagram illustrating syringe sleeve circuitry, according to an embodiment of the present invention.

### DETAILED DESCRIPTION OF THE EMBODIMENTS

Some embodiments of the present invention pertain to an illuminating sleeve configured to slide on, or attach to, a syringe. For purposes of this description, the term “attach to” may include snap onto the syringe, twist or rotate onto the syringe, or connect to the syringe in any manner that would be appreciated by a person of ordinary skill in the art. The syringe sleeve illuminates when a user is delivering anesthetics to a patient and, in particular, delivering the anesthetic to the gums of the patient's mouth. It should be appreciated that the illuminating sleeve may be applicable to syringes other than anesthetic syringes, as well as other medical devices.

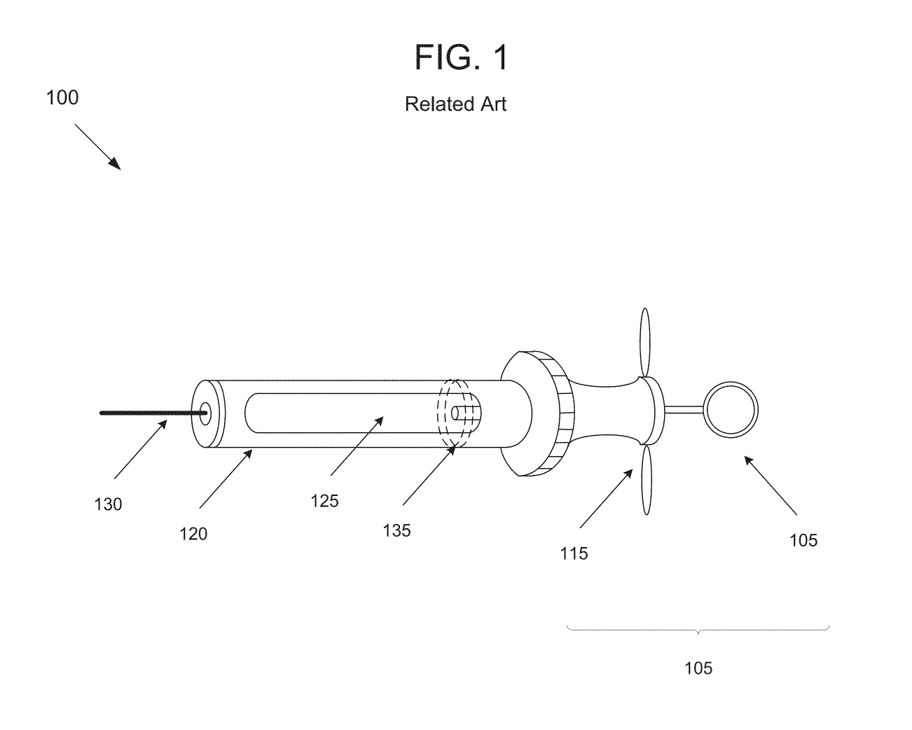

FIG. 1 illustrates a conventional aspirating dental syringe 100. Aspirating dental syringe 100 includes a plunger 105 having a stopper 135 within cylinder 120 to prevent liquid from leaking out of syringe 100. Plunger 105 allows a user to hold syringe 100 and inject the contents thereof. Plunger 105 has a circular loop 110 that allows a user to insert his or her thumb through loop 110 and two additional loops 115 that allow the user insert two of his or her fingers through two additional loops 115.

Aspirating dental syringe 100 also includes a cylinder 120 that contains a liquid, such as Novocain™, Lidocaine, Septocaine, Mepivacaine, a screen 125 allowing the user to view the liquid as the user is applying the liquid to the gums of the patient, and a needle 130 that allows the liquid to be inserted into the gums of the patient.

As discussed above, without adequate light, the user may not be as precise when injecting the liquid into the gums of the patient. For example, he or she may miss the target area of the gums due to a shadow created by the light behind the user. This requires the user to perform the procedure for a subsequent time, potentially causing additional pain to the patient and potentially increasing the area of numbness that the patient will experience after treatment.

Thus, a sleeve having lighting devices that can be attached to one, multiple, or any type syringe may be beneficial. Because the illuminating sleeve may be configured to be an add-on to the conventional syringe, conventional syringes would not need to be replaced, increasing the likelihood of users adopting the technology, and reducing the cost over replacing conventional syringes with syringes having lighting devices.

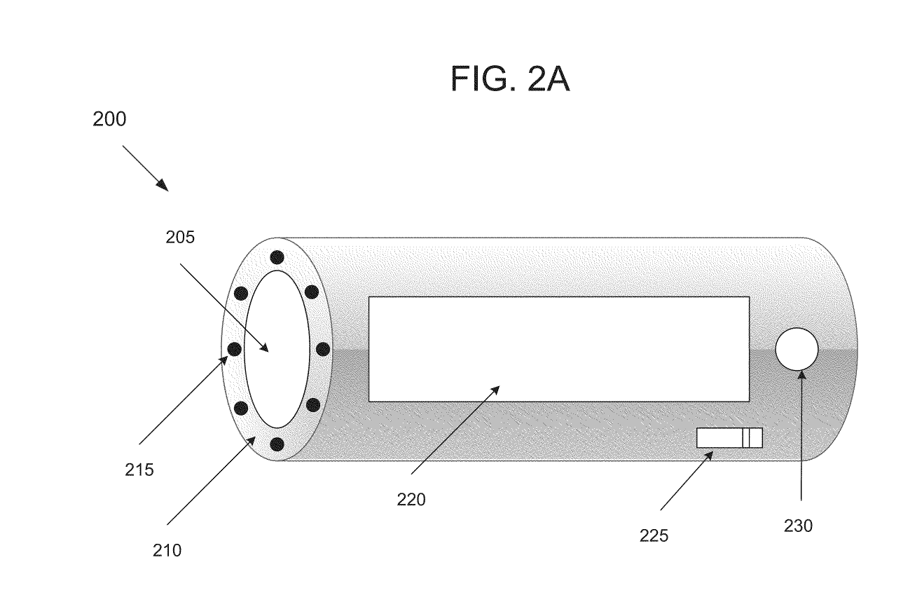

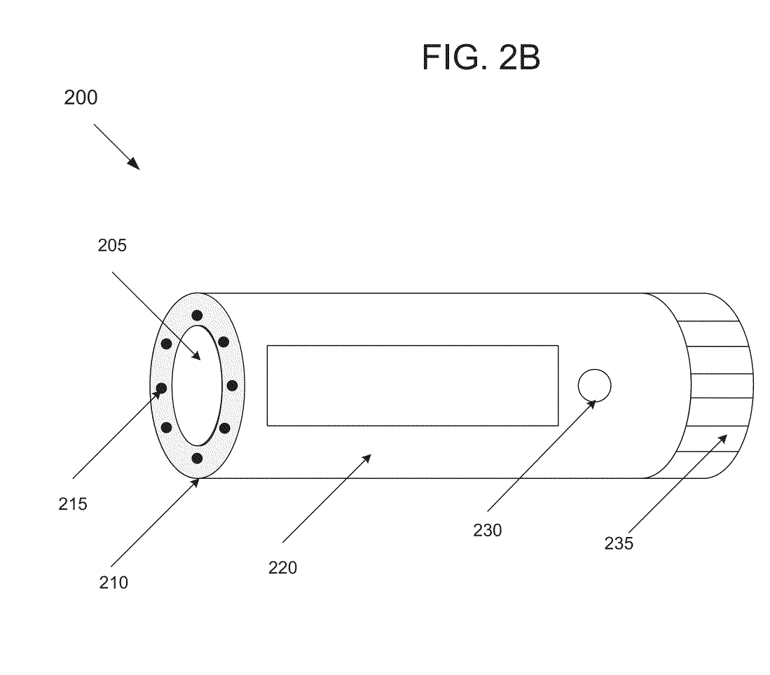

FIG. 2A illustrates a syringe sleeve 200, according to an embodiment of the present invention. In this embodiment, syringe sleeve 200 is a cylindrical tube that is configured to slide on, or attach to, a syringe similar to that shown in FIG. 1. Syringe sleeve 200 may be composed of plastic material, such as polycarbonate material, or any other suitable material. The use of such material may allow for disinfection and easy disposability. Because plastic tends to be relatively inexpensive, syringe sleeve 200 may be a one-time use device. Further, if syringe sleeve 200 is a multiple use device, the use of plastic material may allow for easy disinfection when cleaning the device. Syringe sleeve 200 may also be composed of other non-plastic material, such as stainless steel material, that allows for easy sterilization.

Syringe sleeve 200 may have a predetermined thickness to allow lighting devices 215 to attach to a front end 210 of syringe sleeve 200. In certain embodiments, lighting devices 215 are embedded within front end 205 of syringe sleeve 200. Lighting devices 215 may illuminate when switch 225 is moved to an on state. It should be appreciated that in some embodiments, switch 225 may be a button that can be pressed to activate or deactivate lighting devices 215. As shown in FIG. 2B, syringe sleeve 200 may include an activation unit 235 in the shape of a circular cylinder. However, it should be appreciated that activation unit 235 is not limited to a shape of a circular cylinder. Activation unit 235 when twisted or rotated in a right to left or left to right direction may activate lighting devices. By activating lighting devices 215 using the activation devices shown in FIG. 2A or 2B, a user is able to precisely locate the target area in the mouth to inject anesthetic, for example.

It should be appreciated that depending on the configuration of syringe sleeve 200, the brightness of lighting devices 215 may be adjusted. For example, if switch 225 includes a button, depending on the number of times the button is pressed, the brightness of lighting devices 215 may be adjusted accordingly. In certain embodiments, although not shown in FIG. 2, syringe sleeve may include two buttons (e.g., a first button and a second button). The first button may be configured to activate lighting devices 215 and increase the brightness of the light each time the first button is pressed. The second button may be configured to decrease the brightness of the light illuminated from lighting devices 215 and, depending on the number of times the second button is pressed, the second button may be configured to deactivate lighting devices 215.

In other embodiments, the brightness of the light may be adjusted depending on the position of switch 225. For example, when switch 225 is in an off state, no light is illuminated from lighting devices 215. However, as switch 225 is moved to an on state, the brightness of the light may increase accordingly. This may allow the user to adjust the brightness as desired when delivering the liquid.

It should be appreciated that lighting devices 215 may include light emitting devices (LEDs), fiber optic bulbs, or any type of light bulb or other light generating device that would be appreciated by a person of ordinary skill in the art. For example, depending on whether syringe sleeve 200 is a one-time use device or a multiple use device, fiber optic bulbs may be used for long term use of the device, while LEDs may be used to decrease cost for disposable devices. Stated differently, the type of lighting used (e.g., LED bulbs, fiber optic bulbs, etc.) in syringe sleeve 200 may depend on the material utilized to fabricate syringe sleeve 200 and the intended application. However, embodiments of the present invention are not limited in the type of lighting device that is used.

A power supply (or source) 230 is provided in this embodiment to supply power to lighting devices 215 when syringe sleeve 200 is activated, i.e., when switch 225 is moved to an on state. In some embodiments, switch 225 and power supply 230 may be located at the same location. This allows syringe sleeve 200 to be activated when switch 225 is pressed or moved to an on state allowing power to be supplied from power supply 230 to lighting device 215.

It should be appreciated that power supply 230 may include a button cell battery or any type of battery that would be appreciated by a person of ordinary skill in the art. Power supply 230 may also be a 3 volt battery, a 5 volt battery, or may have any amount of voltage that is sufficient to power syringe sleeve 200. It should be appreciated that power supply 230 may also be a replaceable power supply or a rechargeable power supply, such as a nickel cadmium or lithium ion battery.

In certain embodiments, at least one secondary power supply or other additional power supplies (not shown) may also be provided to supply sufficient power to operate syringe sleeve 200. This may be beneficial when the procedure of injecting the liquid is time consuming. The secondary power supply may also act as a recharging mechanism to recharge first power supply 230.

In certain embodiments, syringe sleeve 200 may also include a universal serial bus (USB) port to charge/recharge power supply 230 and the secondary power supply, if implemented in syringe sleeve 200. Recharging of power supply 230 may mitigate the need for the user to change power supply 230, increasing long term savings.

Although not illustrated in the drawings, syringe sleeve 200 may include, in certain embodiments, position sensors and a processor. The processor may cause the position sensors to record position of the sensor when injecting the liquid. A media recorder (not shown) may also be included on syringe sleeve 200. The media recorder may be located in proximity to lighting devices 215. The media recorder may record video when the liquid is injected. The processor may then transfer the recorded data (i.e., position of the sensor and/or recorded video) to another computing device when the other computing device is connected to syringe sleeve 200 via universal serial bus. This may be beneficial when teaching of anesthetic administration administration to novices, such as interns.

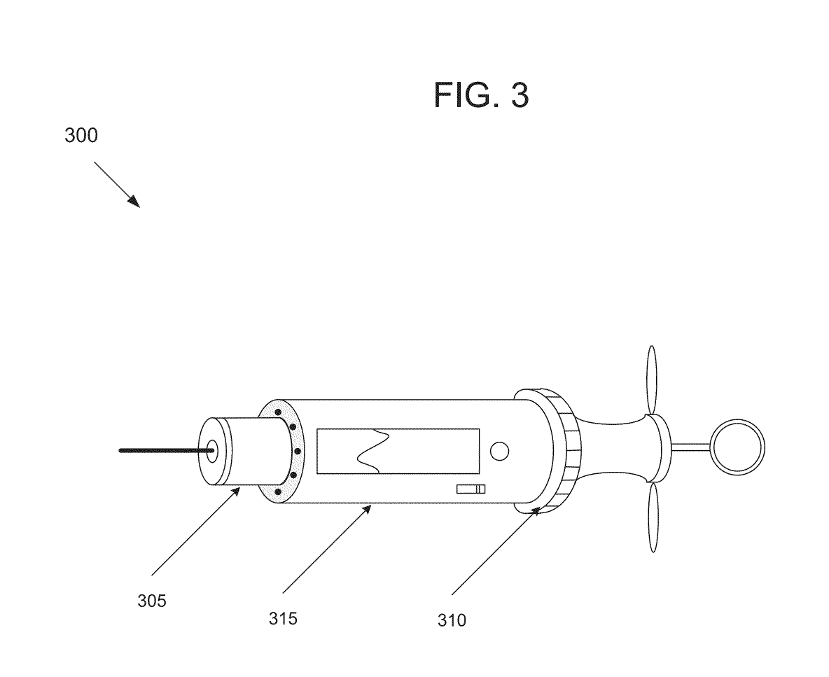

Syringe sleeve 200 also includes a hollow cavity 205. Hollow cavity 205 is configured allow syringe sleeve 200 to attach to, or slide on to, a conventional syringe. For example, as shown in FIG. 3, syringe 305 may be fitted with a syringe sleeve 315. Syringe sleeve 315 may slide up to holding mechanism 310. It should be appreciated that there may be a sufficient amount of friction to maintain the position of syringe sleeve 315 once syringe sleeve 315 is slid onto, or otherwise attached to, syringe 305.

Returning to FIG. 2, as discussed above, conventional syringes generally include a plastic screen or film that allows the user to view the amount of liquid remaining during the medical procedure. See, for example, screen 125 in FIG. 1. In order for the user to view the remaining amount of liquid in the syringe, syringe sleeve 200 includes an opening 220. Opening 220 may allow the user to view how much liquid (e.g., anesthetic) is remaining through the screen of the syringe. This way, the user can control the amount of liquid that is injected into the gums of a patient's mouth, as well as see whether any blood is drawn into the syringe.

In some embodiments, opening 220 may include a film (or screen), and in other embodiments, opening 220 may not include any type of film. The film may be composed of clear plastic material, glass/ceramic material, or any type of clear material that would be appreciated by a person of ordinary skill in the art. By including the film, measurement units (or other suitable metrics) may be shown on the plastic film. In embodiments where the film is not provided, opening 220 may include measurement markers on the border of opening 220.

This allows the user to easily monitor and record how much liquid was injected into the patient. This also eliminates the need for the user to estimate how much liquid was provided to the patient. This may provide the user with the ability to swap out the cartridge from the syringe with another cartridge when delivering the liquid to the patient.

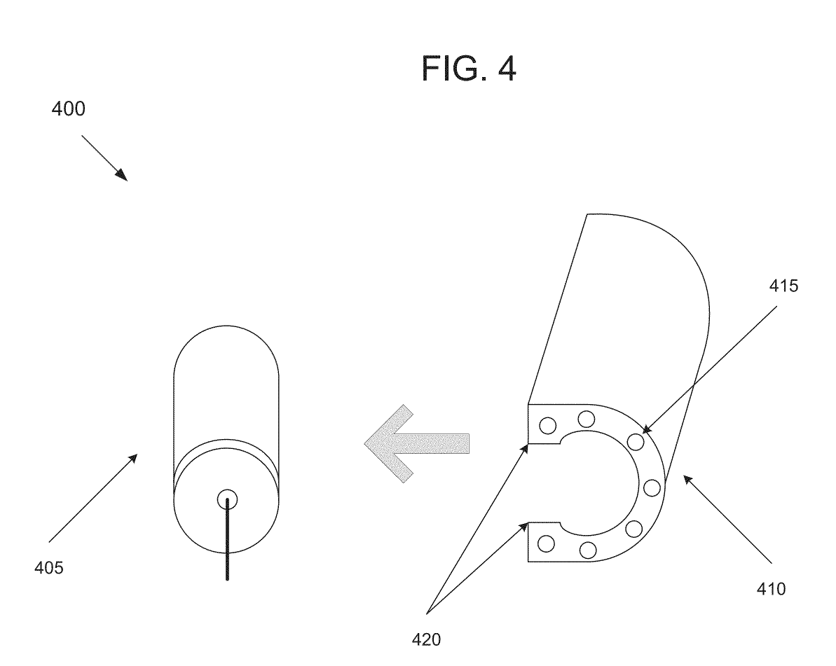

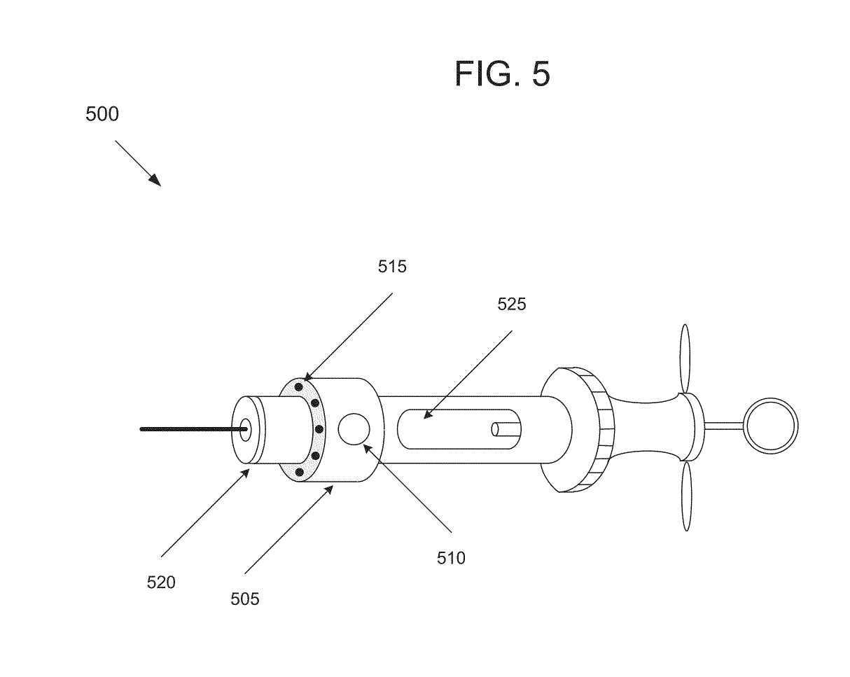

It should be appreciated that embodiments of the syringe sleeve may include various design features and configurations, such as a partial syringe sleeve or a half syringe sleeve. FIG. 4 illustrates a partial (i.e., partially open) syringe sleeve system 400, according to an embodiment of the present invention. In particular, FIG. 4 illustrates a partial syringe sleeve 410 that can be attached to a syringe 405. As shown in FIG. 4, the front end of syringe sleeve 410 includes a plurality of lighting devices 415 configured to illuminate when activate and placed within the mouth of the patient. In order to securely attach syringe sleeve 410 to syringe 405, syringe sleeve 410 includes attachment mechanisms 420. Attachment mechanism 420 may be hook-like in some embodiments to securely attach to partial syringe sleeve 410, but any suitable attachment mechanism may be used. It should be appreciated partial syringe sleeve 410 may include all of the components described above in FIG. 2 or described below with respect to FIG. 5.

FIG. 5 illustrates a syringe sleeve system 500 having a half (or smaller) syringe sleeve 505, according to an embodiment of the present invention. In this embodiment, half syringe sleeve 505 may be positioned near the distal end of syringe 520. The reduced size of half syringe sleeve 505 may further reduce cost. The benefit of half syringe sleeve 505 would reduce cost of production, as well as allow easy removal of the half syringe sleeve 505. Half syringe sleeve 505 may also be rotatable improving visibility of injection site on the right and left sides of the mouth. It should also be appreciated that the location of half syringe sleeve 505 is situated on syringe 520 such that screen 525 remains visible to the user. This allows the user, for example, to note the dosage provided to the patient and how much liquid is left within syringe 520 when delivering the liquid to the patient.

Half syringe sleeve 505 may include lighting devices 515 that can be activated when a switch (or button) 510 is pressed. Because half syringe sleeve 505 may have limited room for circuitry, the location of switch 510 and power supply is the same in this embodiment. For instance, when switch 510 is pressed, the pressing action may cause the button to trigger an activation/deactivation switch (not shown) within half syringe sleeve 505. This causes lighting devices 515 to illuminate.

As discussed above with respect to FIG. 2, switch 510 may be pressed a plurality of times in order to increase the brightness. For example, when lighting devices 515 are illuminated initially, the brightness of lighting devices 515 may be relatively dim. Based on the number of times switch 510 is pressed, the brightness from lighting devices 515 may be increased. A similar operation can be performed to decrease the brightness of lighting devices 515 when lighting devices 515 are fully illuminating upon initial activation.

Further, after pressing switch 510 a predefined number of times, lighting devices 515 may be deactivated. For example, after switch 510 is pressed four times, lighting devices 515 may deactivate. It should be noted that the number of times to press switch 510 to increase or decrease the brightness of lighting devices 515, and the number of times to press switch 510 to deactivate lighting devices 515, may be preset in half syringe sleeve 505. However, the preset configuration may be altered as desired by the user in some embodiments.

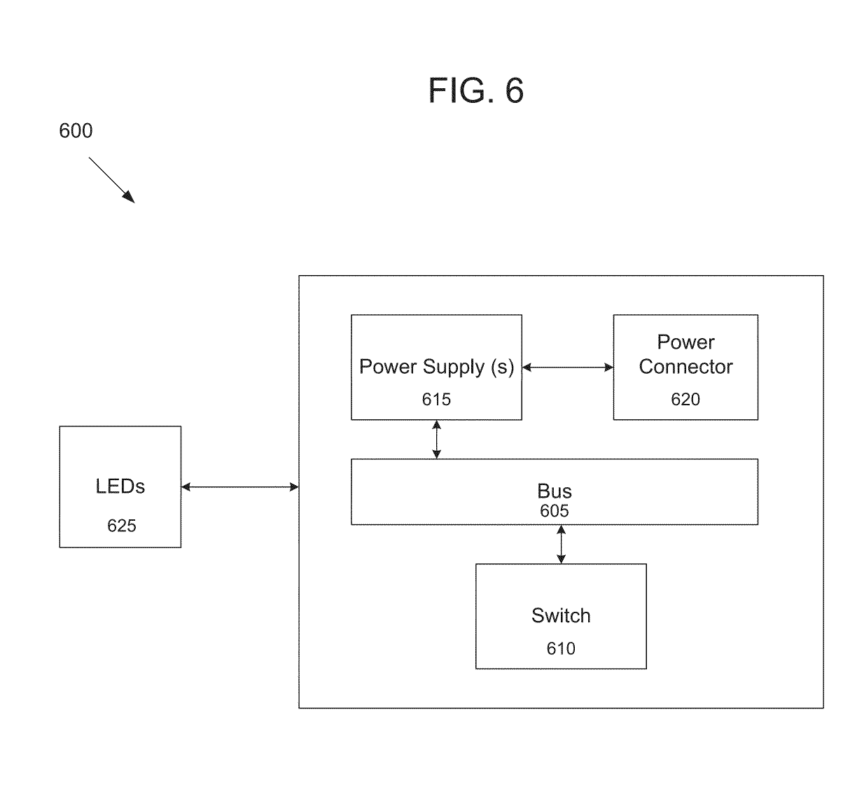

FIG. 6 is a block diagram illustrating syringe sleeve circuitry 600, according to an embodiment of the present invention. It should be appreciated that syringe sleeve circuitry 600 shown in FIG. 6 may be applied to the syringe sleeves shown in FIGS. 2, 4, and/or 5 in some embodiments. In FIG. 6, syringe sleeve circuitry 600 includes a communication bus 605 that is coupled to a switch 610 and at least one power supply 615. For example, when switch 610 is pressed, a signal may be transmitted via communication bus 605 to power supply 615 to activate LEDs 625. Also connected to at least one power supply 615 is a power connector 620. Power connector 620 may be a USB port that allows a USB cable to connect to the USB port to recharge at least one power supply 615 when power is low.

It should be appreciated that embodiments of the present invention pertain to an illuminating cover for a syringe. The cover may be configured to attach to, or slide on, the syringe prior to use. The illuminating cover may include illuminating devices near the distal end of the syringe to allow a user of the syringe to have a brighter view of, for example, internal areas of a patient's mouth.

It will be readily understood that the components of the invention, as generally described and illustrated in the figures herein, may be arranged and designed in a wide variety of different configurations. Thus, the detailed description of the embodiments is not intended to limit the scope of the invention as claimed, but is merely representative of selected embodiments of the invention.

The features, structures, or characteristics of the invention described throughout this specification may be combined in any suitable manner in one or more embodiments. For example, the usage of “certain embodiments,” “some embodiments,” or other similar language, throughout this specification refers to the fact that a particular feature, structure, or characteristic described in connection with an embodiment may be included in at least one embodiment of the invention. Thus, appearances of the phrases “in certain embodiments,” “in some embodiments,” “in other embodiments,” or other similar language, throughout this specification do not necessarily all refer to the same embodiment or group of embodiments, and the described features, structures, or characteristics may be combined in any suitable manner in one or more embodiments.

One having ordinary skill in the art will readily understand that the invention as discussed above may be practiced with steps in a different order, and/or with hardware elements in configurations that are different than those which are disclosed. Therefore, although the invention has been described based upon these preferred embodiments, it would be apparent to those of skill in the art that certain modifications, variations, and alternative constructions would be apparent, while remaining within the spirit and scope of the invention. In order to determine the metes and bounds of the invention, therefore, reference should be made to the appended claims.

1. An apparatus, comprising:
a hollow tube configured to slide on, or attach to, a syringe; and
a plurality of lighting devices proximate to a distal end of the hollow tube, the plurality of lighting devices configured to illuminate when the apparatus is activated.

2. The apparatus of claim 1, wherein the plurality of lighting devices comprise a plurality of fiber optic bulbs and the hollow tube comprises stainless steel material.

3. The apparatus of claim 1, wherein the plurality of lighting devices comprise a plurality of light emitting devices and the hollow tube comprises plastic material.

4. The apparatus of claim 1, further comprising:
a switch configured to illuminate the plurality of lighting devices and deactivate the plurality of light emitting devices.

5. The apparatus of claim 4, wherein the switch is further configured to modify an intensity of illumination emitted from the plurality of lighting devices depending on a position of the switch.

6. The apparatus of claim 1, further comprising:
a first power supply configured to supply power to the plurality of illuminating devices.

7. The apparatus of claim 6, wherein the power supply comprises a replaceable power supply, a rechargeable power supply, or both.

8. The apparatus of claim 6, further comprising:
a secondary power supply configured to supply power to the plurality of illuminating devices, supply power to recharge the first power supply, or both.

9. The apparatus of claim 6, further comprising:
a power connector configured to receive power from an external source to recharge power in the first power supply.

10. The apparatus of claim 1, wherein the hollow tube comprises an opening configured to allow a user to view a level of liquid contained within the syringe.

11. The apparatus of claim 10, wherein the opening comprises measurement markers configured to provide an indication of the level of liquid contained within the syringe.

12. An apparatus, comprising:
a sleeve configured to attach to, or slide on to, a syringe; and
a plurality of lighting devices configured to illuminate when the sleeve is activated.

13. The apparatus of claim 12, wherein the plurality of lighting devices comprise a plurality of fiber optic bulbs and the hollow tube comprises stainless steel material.

14. The apparatus of claim 12, wherein the plurality of lighting devices comprise a plurality of light emitting devices and the hollow tube comprises plastic material.

15. The apparatus of claim 12, further comprising:
an activation/deactivation button configured to activate and deactivate the plurality of lighting devices.

16. The apparatus of claim 15, wherein the switch is further configured to modify an amount of illumination emitted from the plurality of lighting devices depending on a number of times the activation/deactivation button is pressed when the plurality of lighting devices are activated.

17. The apparatus of claim 12, further comprising:
a first power supply configured to supply power to the plurality of illuminating devices.

18. The apparatus of claim 17, wherein the power supply comprises a replaceable power supply, a rechargeable power supply, or both.

19. The apparatus of claim 17, further comprising:
a secondary power supply configured to supply power to the plurality of illuminating devices, supply power to recharge the first power supply, or both.

20. The apparatus of claim 17, further comprising:
a power connector configured to receive power from an external source to recharge power in the first power supply.
